(19) United States
(12) Patent Application Publication
(10) Pub. No.: US 2006/0078592 A1
(43) Pub. Date: Apr. 13, 2006

(54) Drug delivery systems

(75) Inventors: Jay F. Kunzler, Canandaigua, NY (US); Dharmendra M. Jani, Fairport, NY (US); Joseph C. Salamone, Fairport, NY (US)

Correspondence Address: Bausch & Lomb Incorporated, One Bausch & Lomb Place, Rochester, NY, 14604-2701, US

(73) Assignee: Bausch & Lomb Incorporated

(21) Appl. No.: 11/210,124

(22) Filed: Aug. 23, 2005

Related U.S. Application Data
(60) Provisional application No. 60/618,038, filed on Oct. 12, 2004.

(51) Int. Cl. A61F 2/00

(52) U.S. Cl. 424/427

(57) ABSTRACT

A method for making an ocular drug delivery device involves subjecting the device to a supercritical fluid to remove contaminants from the device. The supercritical fluid includes supercritical carbon dioxide. The contaminants include unreacted monomers or oligomers present in the polymeric material used to form the device.

This application claims the benefit under 35 USC 119(e) of Provisional Patent Application No. 60/618,038, filed Oct. 12, 2004.

### FIELD OF THE INVENTION

This invention relates to drug delivery systems for ocular drug delivery, such as a device placed or implanted in the eye to release a pharmaceutically active agent to the eye. Particularly, this invention provides improved methods of making such devices.

### BACKGROUND OF THE INVENTION

Various drugs have been developed to assist in the treatment of a wide variety of ailments and diseases. However, in many instances, such drugs cannot be effectively administered orally or intravenously without the risk of detrimental side effects. Additionally, it is often desired to administer a drug locally, i.e., to the area of the body requiring treatment. Further, it may be desired to administer a drug locally in a sustained release manner, so that relatively small doses of the drug are exposed to the area of the body requiring treatment over an extended period of time.

Accordingly, various sustained release drug delivery devices have been proposed for placing in the eye and treating various eye diseases. Examples are found in the following patents, the disclosures of which are incorporated herein by reference: US 2002/0086051A1 (Viscasillas); US 2002/0106395A1 (Brubaker); U.S. Pat. No. 6,756,049 (Brubaker et al.); U.S. Pat. No. 6,756,058 (Brubaker et al.); US 2002/0110635A1 (Brubaker et al.); U.S. Pat. No. 5,378,475 (Smith et al.); U.S. Pat. No. 5,773,019 (Ashton et al.); U.S. Pat. No. 5,902,598 (Chen et al.); U.S. Pat. No. 6,001,386 (Ashton et al.); U.S. Pat. No. 6,217,895 (Guo et al.); U.S. Pat. No. 6,375,972 (Guo et al.); U.S. patent application Ser. No. 10/403,421 (Drug Delivery Device, filed Mar. 28, 2003) (Mosack et al.); and US 2004/0265356A1 (Mosack).

Many of these devices contain a pharmaceutically active agent and a polymeric material, such as silicone or other hydrophobic materials. As an example, such devices may include an inner drug core including the active agent mixed with a permeable polymeric material, and some type of holder made of a polymeric material impermeable to passage of the active agent. Another example is a matrix of the active agent and a polymeric material.

Various prior methods of making these types of devices involve the step of extracting the polymeric material to remove impurities such as unreacted monomers or oligomers therefrom. The extraction process is important to ensure the device does not leach such impurities once introduced to eye tissue. Extraction is especially important for silicone polymeric materials, as unreacted monomers or oligomers of silicone may be non-biocompatible (for example, irritating to eye tissue or even toxic). A typical method of extracting such polymers employs isopropanol, or other liquid polar solvents, as the extracting material. Accordingly, it is necessary to perform the extraction prior to combining the polymeric material with the active agent. This invention recognized that it would be desirable to perform extraction after combining the active agent with the polymeric material, and this invention provides a method that permits extraction of the device after combining the active agent with the polymeric material.

### BRIEF DESCRIPTION OF THE DRAWINGS

FIG. 1 is a perspective view of a first embodiment of a drug delivery device of this invention.

FIG. 2 is a cross-sectional view of the device of FIG. 1.

FIG. 3 is a cross-sectional view of the device of FIGS. 1 and 2 during assembly.

### SUMMARY OF THE INVENTION

This invention relates to a method for making an ocular drug delivery device, comprising: providing a drug delivery device comprising a polymeric material and a pharmaceutically active agent, said polymeric material including contaminants, and said drug delivery device being sized and configured for implantation or injection in eye tissue; and subjecting the device to a supercritical fluid to remove the contaminants. The contaminants may include unreacted monomers and oligomers. The polymeric material may be a silicone-containing polymer, such as a fluorosilicone-containing polymer or a silicone-containing hydrogel copolymer. A preferred supercritical fluid comprises supercritical carbon dioxide.

The device may comprise a drug core that includes the active agent, and a holder comprising the polymeric material, wherein the drug core is held in the holder. The device may comprise a matrix of the polymeric material and the pharmaceutically active agent. Preferably, the drug delivery device comprises a pharmaceutically active salt, and the contaminants are hydrophobic, such as silicone-containing unreacted monomers and oligomers.

### DETAILED DESCRIPTION OF PREFERRED EMBODIMENTS

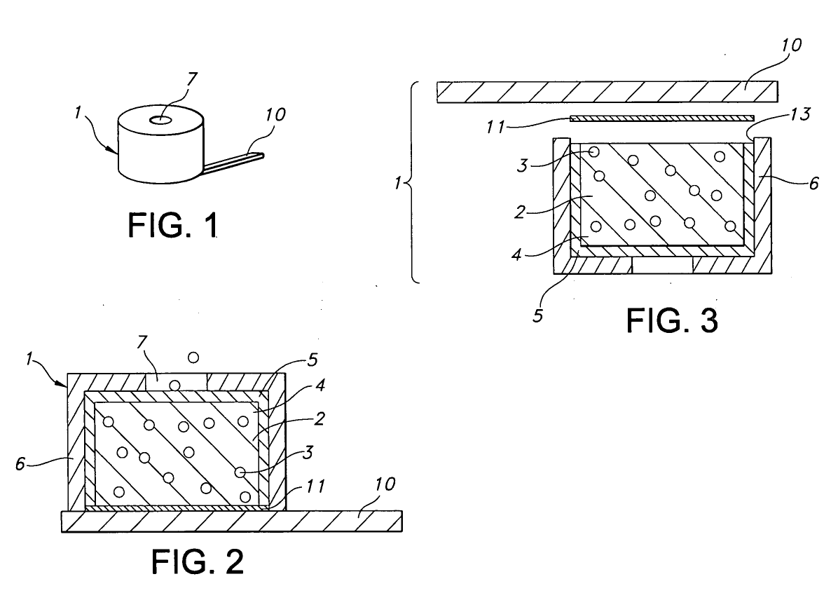

FIGS. 1 and 2 illustrate a first embodiment of a device of this invention. Device 1 is a sustained release drug delivery device for implanting in the eye. Device 1 includes inner drug core 2 including a pharmaceutically active agent 3.

This active agent may include any compound, composition of matter, or mixture thereof that can be delivered from the device to produce a beneficial and useful result to the eye, especially an agent effective in obtaining a desired local or systemic physiological or pharmacological effect. Examples of such agents include: anesthetics and pain killing agents such as lidocaine and related compounds and benzodiazepam and related compounds; anti-cancer agents such as 5-fluorouracil, adriamycin and related compounds; anti-fungal agents such as fluconazole and related compounds; anti-viral agents such as trisodium phosphomonoformate, trifluorothymidine, acyclovir, ganciclovir, DDI and AZT; cell transport/mobility impending agents such as colchicine, vincristine, cytochalasin B and related compounds; antiglaucoma drugs such as beta-blockers: timolol, betaxolol, atenalol, etc; antihypertensives; decongestants such as phenylephrine, naphazoline, and tetrahydrazoline; immunological response modifiers such as muramyl dipeptide and related compounds; peptides and proteins such as cyclosporin, insulin, growth hormones, insulin related growth factor, heat shock proteins and related compounds; steroidal compounds such as dexamethasone, prednisolone and related compounds; low solubility steroids such as fluocinolone acetonide and related compounds; carbonic anhydrase inhibitors; diagnostic agents; antiapoptosis agents; gene therapy agents; sequestering agents; reductants such as glutathione; antipermeability agents; antisense compounds; antiproliferative agents; antibody conjugates; antidepressants; bloodflow enhancers; antiasthmatic drugs; antiparasiticagents; non-steroidal anti inflammatory agents such as ibuprofen; nutrients and vitamins: enzyme inhibitors: antioxidants; anticataract drugs; aldose reductase inhibitors; cytoprotectants; cytokines, cytokine inhibitors, and cytokin protectants; uv blockers; mast cell stabilizers; and anti neovascular agents such as antiangiogenic agents like matrix metalloprotease inhibitors.

Examples of such agents also include: neuroprotectants such as nimodipine and related compounds; antibiotics such as tetracycline, chlortetracycline, bacitracin, neomycin, polymyxin, gramicidin, oxytetracycline, chloramphenicol, gentamycin, and erythromycin; antiinfectives; antibacterials such as sulfonamides, sulfacetamide, sulfamethizole, sulfisoxazole; nitrofurazone, and sodium propionate; antiallergenics such as antazoline, methapyriline, chlorpheniramine, pyrilamine and prophenpyridamine; anti-inflammatories such as hydrocortisone, hydrocortisone acetate, dexamethasone 21-phosphate, fluocinolone, medrysone, methylprednisolone, prednisolone 21-phosphate, prednisolone acetate, fluoromethalone, betamethasone and triminolone; miotics and anti-cholinesterase such as pilocarpine, eserine salicylate, carbachol, di-isopropyl fluorophosphate, phospholine iodine, and demecarium bromide; mydriatics such as atropine sulfate, cyclopentolate, homatropine, scopolamine, tropicamide, eucatropine, and hydroxyamphetamine; svmpathomimetics such as epinephrine; and prodrugs such as those described in Design of Prodrugs, edited by Hans Bundgaard, Elsevier Scientific Publishing Co., Amsterdam, 1985. In addition to the above agents, other agents suitable for treating, managing, or diagnosing conditions in a mammalian organism may be placed in the inner core and administered using the sustained release drug delivery devices of the current invention. Once again, reference may be made to any standard pharmaceutical textbook such as Remington's Pharmaceutical Sciences for the identity of other agents.

Any pharmaceutically acceptable form of such a compound may be employed in the practice of the present invention, i.e., the free base or a pharmaceutically acceptable salt or ester thereof. Pharmaceutically acceptable salts, for instance, include sulfate, lactate, acetate, stearate, hydrochloride, tartrate, maleate and the like.

As shown in the illustrated embodiment, active agent 3 may be mixed with a polymeric material 4. Material 4 is a polymeric material that is compatible with body fluids and the eye. Additionally, this material should be permeable to passage of the active agent 3 therethrough, particularly when the device is exposed to body fluids. For the illustrated embodiment, this polymeric material is poly(vinyl alcohol) (PVA). Also, in this embodiment, inner drug core 2 may be coated with a coating 5 of additional polymeric material which may be the same or different from material 4 mixed with the active agent. For the illustrated embodiment, the coating 5 employed is also PVA.

Device 1 includes a holder 6 for the inner drug core 2. Holder 6 is made of a material that is impermeable to passage of the active agent 3 therethrough. Since holder 6 is made of the impermeable material, at least one passageway 7 is formed in holder 6 to permit active agent 3 to pass therethrough and contact eye tissue. In other words, active agent passes through any permeable material 4 and permeable coating 5, and exits the device through passageway 7. For the illustrated embodiment, the holder is made of silicone, especially polydimethylsiloxane (PDMS) material.

A prior method of making a device of the type shown in FIGS. 1 and 2 includes the following procedures. A cylindrical cup of silicone is separately formed, for example by molding, having a size generally corresponding to the drug core tablet and a shape as generally shown in FIG. 2. This silicone holder is then extracted with a solvent such as isopropanol. Openings 7 are placed in the silicone holder, for example, by boring or with the laser. A drop of liquid PVA is placed into the holder through the open end 13 of the holder, this open end best seen in FIG. 3. Then, the inner drug core tablet is placed into the silicone holder through the same open end 13 and pressed into the cylindrical holder. As a result, the pressing of the tablet causes the liquid PVA to fill the space between the tablet inner core and the silicone holder, thus forming permeable layer 5 shown in FIGS. 1 and 2. For the illustrated embodiment, a layer of adhesive 11 is applied to the open end 13 of the holder to fully enclose the inner drug core tablet at this end. Suture tab 10 is inserted at this end of the device. The liquid PVA and adhesive are cured by heating the assembly.

As mentioned, this invention recognized that it would be more desirable to extract the device after loading the device with the pharmaceutically active agent. Particularly, the device holder is extracted to remove residual materials therefrom. For example, in the case of a silicone holder, the holder may include lower molecular weight materials such as unreacted monomeric material and oligomers. Such materials may irritate eye tissue. Also, the presence of such residual materials may also deleteriously affect adherence of the holder surfaces.

It is noted that traditional extracting solvents do not lend themselves to extracting devices already containing pharmaceutically active agent, as relatively large amounts of various pharmaceutically active agents would be dissolved in and removed by isopropanol and similar solvents. Also, it is noted this invention does not rely on supercritical fluid to disperse the active agent in the device polymeric material, rather, extraction with the supercritical fluid does not need to occur until after the device polymeric material is loaded with the active agent.

As mentioned, any pharmaceutically acceptable form of the pharmaceutically active agent may be employed in this invention. However, many supercritical fluids, including supercritical carbon dioxide, are relatively hydrophobic. Thus, the supercritical fluid better dissolves hydrophobic material. Accordingly, this invention is particularly useful in extracting hydrophobic contaminants, such as silicone-containing oligomers or unreacted monomers. Additionally, the salt forms of various pharmaceutically active agents are relatively hydrophobic. Accordingly, this invention is particularly useful in extracting devices containing pharmaceutically active salts, in that the active salts are not readily dissolved in, nor removed from the device by, the treatment with supercritical fluid.

### Example 1

A method of making a device of the type shown in FIGS. 1 and 2, according to this invention, includes the following procedures. The cylindrical cup of silicone is separately formed, for example by molding, having a size generally corresponding to the drug core tablet and a shape as generally shown in FIG. 2. The openings 7 are placed in the silicone holder, for example, by boring or with the laser. The drop of liquid PVA is placed into the holder through the open end 13 of the holder, this open end best seen in FIG. 3. Then, the inner drug core tablet is placed into the silicone holder through the same open end 13 and pressed into the cylindrical holder. If desired, a layer of adhesive 11 is applied to the open end 13 of the holder to fully enclose the inner drug core tablet at this end. Tab 10 is inserted at this end of the device. The liquid PVA and adhesive are cured by heating the assembly. Following these procedures, the device is extracted with supercritical fluid, such as supercritical carbon dioxide. The exposure to supercritical fluid removes contaminants, including unreacted monomers or oligomers present in the silicone cup. Additionally, the supercritical fluid will remove various contaminants resulting from the procedures of placing the openings in the holder.

In addition to the materials illustrated in Example 1, a wide variety of materials may be used to construct the devices of the present invention. The only requirements are that they are inert; non-immunogenic and of the desired permeability. Materials that may be suitable for fabricating the device include naturally occurring or synthetic materials that are biologically compatible with body fluids and body tissues, and essentially insoluble in the body fluids with which the material will come in contact. The use of rapidly dissolving materials or materials highly soluble in body fluids are to be avoided since dissolution of the wall would affect the constancy of the drug release, as well as the capability of the device to remain in place for a prolonged period of time.

Naturally occurring or synthetic materials that are biologically compatible with body fluids and eye tissues and essentially insoluble in body fluids which the material will come in contact include, but are not limited to, glass, metal, ceramics, poly(vinyl acetate), crosslinked poly(vinyl alcohol), insolubilized poly(vinyl alcohol), crosslinked poly(vinyl butyrate), ethylene-ethyl acrylate copolymer, poly(2-ethylhexyl acrylate), poly(vinyl chloride), poly(vinyl acetal), plasiticized ethylene-vinyl acetate copolymer, ethylene-vinyl chloride copolymer, poly(vinyl alcohol), polyvinyl esters, polyvinylbutyrate, poly(vinyl formal), polyamides, poly(methyl methacrylate), poly(butyl methacrylate), plasticized poly(vinyl chloride), plasticized nylon, plasticized poly(ethylene terephthalate), natural rubber, polyisoprene, polyisobutylene, polybutadiene, polyethylene, polytetrafluoroethylene, poly(vinylidene chloride), polyacrylonitrile, crosslinked poly(N-vinylpyrrolidone), polytrifluorochloroethylene, chlorinated polyethylene, poly(1,4′-isopropylidene diphenylene carbonate), vinylidene chloride-acrylonitrile copolymer, vinyl chloride-diethyl fumarate copolymer, butadiene/styrene copolymers, silicone rubbers, especially the medical grade polydimethylsiloxanes, fluorosilicone polymers, perfouoropolyethers, ethylene-propylene rubber, silicone-carbonate copolymers, and vinylidene chloride-vinyl chloride copolymer.

The illustrated embodiment includes a tab 10 which may be made of a wide variety of materials, including those mentioned above for the permeable polymeric material and/or the holder. Tab 10 may be provided in order to attach the device to a desired location in the eye, for example, by suturing. For the illustrated embodiment, tab 10 is made of PVA and is adhered to the inner drug core 2 with adhesive 11. Adhesive 11 may be a curable silicone adhesive, a curable PVA solution, or the like.

It will be appreciated the dimensions of the device can vary with the size of the device, the size of the inner drug core, and the holder that surrounds the core or reservoir. The physical size of the device should be selected so that it does not interfere with physiological functions at the implantation site of the mammalian organism. The targeted disease state, type of mammalian organism, location of administration, and agents or agent administered are among the factors which would effect the desired size of the sustained release drug delivery device. However, because the device is intended for placement in the eye, the device is relatively small in size. Generally, it is preferred that the device, excluding the suture tab, has a maximum height, width and length each no greater than 10 mm, more preferably no greater than 5 mm, and most preferably no greater than 3 mm.

The process of this invention is applicable to many other configurations of the device, besides the illustrated embodiment. Many other configurations of such devices are known in the art.

According to other embodiments of this invention, the device comprises a solid matrix of the polymeric material and the pharmaceutically active agent. This matrix material may be formed into a desired shape, such as a film, sphere, cylinder or lens-shaped article. The resultant device may be implanted surgically in the eye. For example, the drug delivery device may be implanted below the sclera. Alternately, the device may be implanted by injecting the device into the eye. For example, a sphere- or cylinder-shaped device may be inserted into the vitreous through a 0.5-mm opening in the sclera provided by a TSV-25 cannula. Generally, the active agent is included in the matrix in an amount of 0.1 to 10% (w/w), more preferably, 1 to 5% (w/w), based on total weight of the matrix.

As a first example, the polymeric material may be a silicone hydrogel loaded with the pharmaceutically active agent.

A hydrogel is a hydrated crosslinked or insolubilized polymeric system that contains water in an equilibrium state. Hydrogel devices are generally formed by polymerizing a mixture of device-forming monomers including at least one hydrophilic monomer. Hydrophilic device-forming monomers include: unsaturated carboxylic acids such as methacrylic acid and acrylic acid; (meth)acrylic substituted alcohols or glycols such as 2-hydroxyethyl methacrylate, 2-hydroxyethyl acrylate, and glyceryl methacrylate; vinyl lactams such as N-vinyl-2-pyrrolidone; and acrylamides such as methacrylamide and N,N-dimethylacrylamide. Other hydrophilic monomers are well-known in the art.

The monomer mixture generally includes a crosslinking monomer, a crosslinking monomer being defined as a monomer having multiple polymerizable functionalities. One of the hydrophilic monomers may function as a crosslinking monomer or a separate crosslinking monomer may be employed. Representative crosslinking monomers include: divinylbenzene, allyl methacrylate, ethylene glycol dimethacrylate, tetraethyleneglycol dimethacrylate, polyethyleneglycol dimethacrylate, and vinyl carbonate derivatives of the glycol dimethacrylates.

In the case of silicone hydrogels, the device-forming monomer mixture includes, in addition to a hydrophilic monomer, at least one silicone-containing monomer. When the silicone-containing monomer includes multiple polymerizable groups, it may function as the crosslinking monomer. This invention is particularly suited for extraction of silicone hydrogel biomedical devices. Generally, unreacted silicone-containing monomers, and oligomers formed from these monomers, are hydrophobic and more difficult to extract from the polymeric device.

One suitable class of silicone containing monomers include known bulky, monofunctional polysiloxanylalkyl monomers represented by Formula (I):

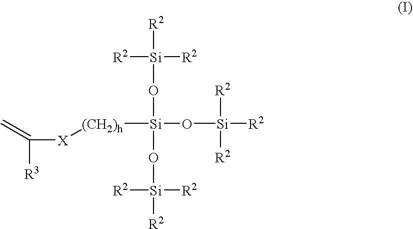

X denotes —COO—, —CONR⁴—, —OCOO—, or —OCONR⁴— where each where R⁴ is H or lower alkyl; R³ denotes hydrogen or methyl; h is 1 to 10; and each R² independently denotes a lower alkyl or halogenated alkyl radical, a phenyl radical or a radical of the formula
—Si(R⁵)₃
wherein each R⁵ is independently a lower alkyl radical or a phenyl radical. Such bulky monomers specifically include 3-methacryloxypropyltris(trimethylsiloxy)silane, pentamethyldisiloxanylmethyl methacrylate, methyldi(trimethylsiloxy)methacryloxymethylsilane, 3-[tris(trimethylsiloxy)silyl]propylvinyl carbamate, and 3-[tris(trimethylsiloxy)silyl]propylvinyl carbonate.

Another suitable class is multifunctional ethylenically “end-capped” siloxane-containing monomers, especially difunctional monomers represented Formula (II): 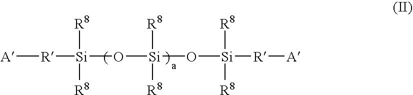
wherein:

each A′ is independently an activated unsaturated group;

each R′ is independently are an alkylene group having 1 to 10 carbon atoms wherein the carbon atoms may include ester, ether, urethane or ureido linkages therebetween;

each R⁸ is independently selected from monovalent hydrocarbon radicals or halogen substituted monovalent hydrocarbon radicals having 1 to 18 carbon atoms which may include ether linkages therebetween, and

a is an integer equal to or greater than 1. Preferably, each R⁸ is independently selected from alkyl groups, phenyl groups and fluoro-substituted alkyl or alkyloxy groups. It is further noted that at least one R⁸ may be a fluoro-substituted alkyl group such as that represented by the formula:
—D′—(CF₂)_{S}—M′
wherein:

D′ is an alkylene group having 1 to 10 carbon atoms wherein said carbon atoms may include ether linkages therebetween;

M′ is hydrogen, fluorine, or alkyl group but preferably hydrogen; and

s is an integer from 1 to 20, preferably 1 to 6.

With respect to A′, the term “activated” is used to describe unsaturated groups which include at least one substituent which facilitates free radical polymerization, preferably an ethylenically unsaturated radical. Although a wide variety of such groups may be used, preferably, A′ is an ester or amide of (meth)acrylic acid represented by the general formula: 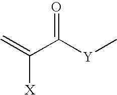
wherein X is preferably hydrogen or methyl, and Y is —O— or —NH—. Examples of other suitable activated unsaturated groups include vinyl carbonates, vinyl carbamates, fumarates, fumaramides, maleates, acrylonitryl, vinyl ether and styryl. Specific examples of monomers of Formula (II) include the following: 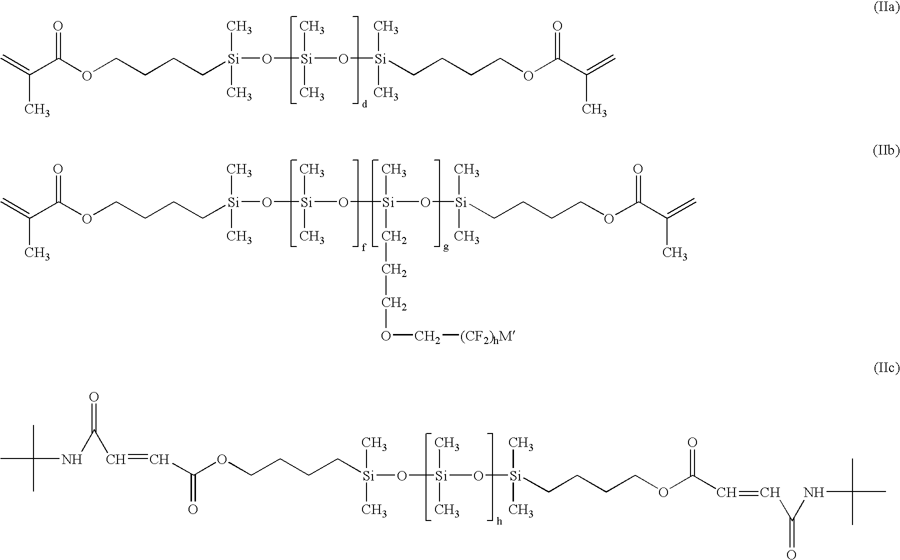
wherein:

d, f, g and k range from 0 to 250, preferably from 2 to 100; h is an integer from 1 to 20, preferably 1 to 6; and

M′ is hydrogen or fluorine.

A further suitable class of silicone-containing monomers includes monomers of the Formulae (IIIa) and (IIIb):
E′(*D*A*D*G)ₐ*D*A*D*E′; (IIIa)
or
E′(*D*G*D*A)ₐ*D*G*D*E′; (IIIb)
wherein:

D denotes an alkyl diradical, an alkyl cycloalkyl diradical, a cycloalkyl diradical, an aryl diradical or an alkylaryl diradical having 6 to 30 carbon atoms;

G denotes an alkyl diradical, a cycloalkyl diradical, an alkyl cycloalkyl diradical, an aryl diradical or an alkylaryl diradical having 1 to 40 carbon atoms and which may contain ether, thio or amine linkages in the main chain;

* denotes a urethane or ureido linkage;

a is at least 1;

A denotes a divalent polymeric radical of the formula: 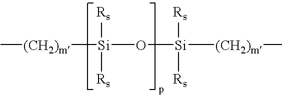
wherein:

each R^{S} independently denotes an alkyl or fluoro-substituted alkyl group having 1 to 10 carbon atoms which may contain ether linkages between carbon atoms;

m′ is at least 1; and

p is a number which provides a moiety weight of 400 to 10,000;

each E′ independently denotes a polymerizable unsaturated organic radical represented by the formula: 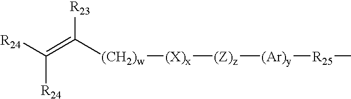
wherein:

R₂₃ is hydrogen or methyl;

R₂₄ is hydrogen, an alkyl radical having 1 to 6 carbon atoms, or a —CO—Y—R₂₆ radical wherein Y is —O—, —S— or —NH—;

R₂₅ is a divalent alkylene radical having 1 to 10 carbon atoms; R₂₆ is a alkyl radical having 1 to 12 carbon atoms; X denotes —CO— or —OCO—; Z denotes —O— or —NH—; Ar denotes an aromatic radical having 6 to 30 carbon atoms; w is 0 to 6; x is 0 or 1; y is 0 or 1; and z is 0 or 1.

A specific urethane monomer is represented by the following: 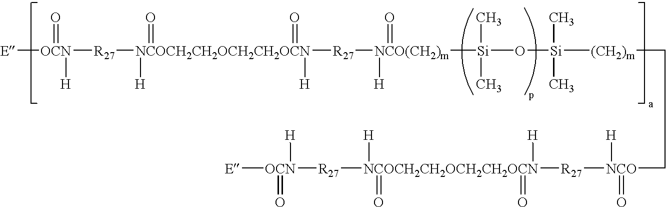
wherein m is at least 1 and is preferably 3 or 4, a is at least 1 and preferably is 1, p is a number which provides a moiety weight of 400 to 10,000 and is preferably at least 30, R₂₇ is a diradical of a diisocyanate after removal of the isocyanate group, such as the diradical of isophorone diisocyanate, and each E″ is a group represented by:

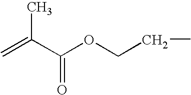

Other silicone-containing monomers include the silicone-containing monomers described in U.S. Pat. Nos. 5,034,461, 5,070,215, 5,260,000, 5,610,252 and 5,496,871, the disclosures of which are incorporated herein by reference. Other silicone-containing monomers are well-known in the art.

These matrices of a silicone hydrogel and active agent may be prepared by mixing the active agent and the device-forming monomeric mixture, including any diluent. Then, this initial mixture is added to a mold providing the final shape and configuration of the solid matrix device. While contained in the mold, the mixture is polymerized by exposure to light energy, such as a UV light source, or a source of visible light in the blue spectrum. Alternately, the mixture may be cured thermally. Finally, the resultant solid matrix device is removed from the mold, and extracted with supercritical fluid.

As a second example, the polymeric material may be a silicone-containing, non-hydrogel polymer loaded with the pharmaceutically active agent. This class of materials include at least one silicone-containing monomer as a device-forming monomer. A crosslinking monomer may also be included in the initial monomeric mixture, although when the silicone-containing monomer includes multiple polymerizable radicals, it may function as the crosslinking monomer. Additionally, this initial monomeric mixture may include a non-silicone hydrophobic co-monomer, such as an alkyl (meth)acrylate or fluoroalkyl (meth)acrylate.

The pharmaceutically active agent is added to the device-forming monomeric mixture, including any diluent, and this initial mixture is added to a mold providing the final shape and configuration of the solid matrix device. While contained in the mold, the mixture is polymerized by exposure to light energy and/or thermal energy. The resultant solid matrix device is removed from the mold and extracted with supercritical fluid. This invention is particularly suited for extraction of silicone non-hydrogel devices due to the presence of unreacted silicone-containing monomers, and oligomers formed from these monomers.

As a third example, the polymeric material may be a non-silicone polymer loaded with the pharmaceutically active agent, such as a fumarate polymer loaded with the pharmaceutically active agent. As an example, these matrices may be prepared by crosslinking polypropylene fumarate (PPF) in the presence of the active agent. More specifically, a mixture is first provided, the mixture including PPF and the active agent. Generally, this initial mixture will further include a co-monomer and/or a solvent. Since PPF is a hydrophobic polymer, a hydrophobic or amphiphilic carrier (co-monomer or solvent) is generally required to dissolve this polymer.

According to preferred embodiments, this initial mixture includes an amphiphilic monomer. Representative amphiphilic monomers include: (meth)acrylic substituted alcohols, such as 2-hydroxyethyl methacrylate, 2-hydroxyethyl acrylate and glycerol methacrylate; vinyl lactams, such as N-vinylpyrrolidone; and (meth)acrylamides, such as methacrylamide and N,N-dimethylacrylamide.

Optionally, this initial mixture may include a hydrophobic co-monomer, either in place of, or in addition to, the amphiphilic co-monomer. Representative hydrophobic comonomers include: a silicone-containing monomer, such as a silicone-containing (meth)acrylate; or an alkyl (meth)acrylate. As an example, a hydrophobic co-monomer will tend to render the resultant solid polymer less permeable to the active agent, whereas a hydrophilic co-monomer more permeable to the active agent. Thus, hydrophobic and hydrophilic co-monomers may be included, at appropriate ratios, to adjust permeability.

When copolymerizing PPF and the co-monomer, the PPF will function as a crosslinking agent, a crosslinking agent being defined as a polymerizable material having multiple polymerizable functionalities. Optionally, a separate crosslinking monomer may be included in the initial monomeric mixture. Examples of crosslinking agents include polyvinyl, typically di- or tri-vinyl monomers, such as di- or tri(meth)acrylates of diethyleneglycol, triethyleneglycol, butyleneglycol and hexane-1,6-diol; divinylbenzene; allylmethacrylate; and bis(4-vinyloxybutyl) adipate.

Preferably, this initial mixture includes a photopolymerization initiator. Typical polymerization initiators include free-radical-generating polymerization initiators of the type illustrated by acetyl peroxide, lauroyl peroxide, decanoyl peroxide, caprylyl peroxide, benzoyl peroxide, tertiary butyl peroxypivalate, sodium percarbonate, tertiary butyl peroctoate, azobis-isobutyronitrile (AIBN); phosphine oxides such as bis(2,4,6-trimethylbenzoyl)phenylphosphine oxide; and acetophenones, such as diethoxyacetophenone.

This initial mixture is added to a mold providing the final shape and configuration of the solid matrix. While contained in the mold, the mixture is polymerized by exposure to light energy, such as a UV light source, or a source of visible light in the blue spectrum. Finally, the resultant solid matrix is removed from the mold.

Alternately, the active agent is combined with a copolymer of PPF, such as a copolymer of PPF and ethylene glycol. Such copolymers are synthesized by esterification of PPF and PEG at a desired molar ratio. For example, a molar ratio of 1:2 PPF:PEG yields a PEG-PPF-PEG triblock copolymer. By adding the active agent to a solution containing the copolymer at the gellation temperature of the copolymer, the copolymer gels and precipitates, forming a matrix with entrapped active agent. Additionally, by selecting a gellation temperature approximating body temperature, such copolymers (or microspheres or nanospheres) will gel upon injection into the body of a patient.

### Example 2

This example illustrates synthesis procedure of methacrylate end-capped polysiloxane with fluorinated side chain.

Synthesis of 1,3-bis(4-methacryloyloxybutyl)tetramethyldisiloxane (M₂). To a 5-liter four-neck resin flask equipped with a mechanical stirrer, Dean-Stark trap, heating mantle, water cooled condenser and thermometer, was added 1,1-dimethyl-1-sila-2-oxacyclohexane (521 g, 4.0 mole), methacrylic acid (361 g, 4.2 mole), and concentrated sulfuric acid (25.5 g, mole). To the reaction mixture was then added 1L of cyclohexane and hydroquinone (0.95 g, 8.6 mmole) as a polymerization inhibitor. The reaction mixture was heated to reflux for five hours during which time 28 mL of water was collected. The reaction mixture was then cooled, divided, and passed through two chromatography columns filled with 1 kg of alumina (packed using cyclohexane as eluant). The cyclohexane was removed using a rotary evaporator and the resultant produce (designated M₂) was placed under vacuum (0.2 mm Hg) for one hour at 80° C. (yield 80%, purity by GC 96%). ¹H-NMR (CDCl₃, TMS, ∂, ppm): 0.1 (s, 12H, Si—CH₃), 0.5 (t, 4H, Si—CH₂—), 1.5-1.8 (m, 8H, Si—CH₂—CH2—CH₂ and Si—CH₂—CH₂—CH₂), 1.95 (s, 6H, ═C—CH₃), 4.1 (t, 4H, —CH₂—O—C(O)), 5.6 (s, 2H, ═C—H), 6.2 (s, 2H, ═C—H).

Synthesis of methacrylate end-capped poly(25 mole % methyl siloxane)-co-(75 mole % dimethylsiloxane)(M₂D₇₅D₂₅H). To a 1000-mL round bottom flask under dry nitrogen was added octamethylcyclotetrasiloxane (D₄) (371.9 g, 1.25 mole), tetramethylcyclotetrasiloxane (D₄H) (100.4 g, 0.42 mole) and M₂ (27.7 g, 0.7 mole). Trifluoromethane sulfonic acid (0.25%, 1.25 g, 8.3 mmole) was added as initiator. The reaction mixture was stirred 24 hours with vigorous stirring at room temperature. Sodium bicarbonate (10 g, 0.119 mole) was then added and the reaction mixture was again stirred for 24 hours. The resultant solution was filtered through a 0.3μ Teflon® filter. The filtered solution was vacuum stripped and placed under vacuum (>0.1 mm Hg) at 50° C. to remove the unreacted silicone cyclics. The resulting silicone hydride functionalized siloxane was a viscous, clear fluid; Yield 70%; SEC: Mn=7,500, Mw/Mn=2.2; ¹H-NMR (CDCl₃, TMS, ∂, ppm): 0.1 (s, 525H, Si—CH₃), 0.5 (t, 4H, Si—CH₂—), 1.5-1.8 (m, 8H, Si—CH₂—CH₂—CH₂ and Si—CH₂—CH₂—CH2), 1.95 (s, 6H, ═C—CH₃), 4.1 (t, 4H, —CH₂—O—C(O)), 4.5 (s, 25H, Si—H), 5.6 (s, 2H, ═C—H), 6.2 (s, 2H, ═C—H).

Synthesis of methacrylate end-capped poly(25 mole % (3-(2,2,3,3,4,4,5,5-octafluoropentoxy)propylmethylsiloxane)-co-(75 mole % dimethylsiloxane). To a 500-mL round bottom flask equipped with a magnetic stirrer and water condenser was added M₂D₇₅D₂₅H (15 g, 0.002 mole), allyloxyoctafluoropentane (27.2 g, 0.1 mole), tetramethyldisiloxane platinum complex (2.5 mL of a 10% solution in xylenes), 75 mL of dioxane and 150 mL of anhydrous tetrahydrofuran under a nitrogen blanket. The reaction mixture was heated to 75° C. and the reaction was monitored by IR and ¹H-NMR spectroscopy for loss of silicone hydride. The reaction was complete in 4 to 5 hours of reflux. The resulting solution was placed on a rotoevaporator to remove tetrahydrofuran and dioxane. The resultant crude product was diluted with 300 mL of a 20% methylene chloride in pentane solution and passed through a 15 gram column of silica gel using a 50% solution of methylene chloride in pentane as eluant. The collected solution was again placed on the rotoevaporator to remove solvent and the resultant clear oil was placed under vacuum (>0.1 mm Hg) at 50° C. for four hours. The resulting octafluoro functionalized side-chain siloxane was a viscous, clear fluid; Yield 65%; SEC: Mn=18,000, Mw/Mn=2.3; ¹H-NMR (CDCl₃, TMS, ∂, ppm): 0.1 (s, 525H, Si—CH₃), 0.5 (t, 54H, Si—CH₂—), 1.5-1.8 (m, 58H, Si—CH₂—CH₂—CH₂ and Si—CH₂—CH₂—CH₂), 1.95 (s, 6H, ═C—CH₃), 4.1 (t, 4H, —CH₂—O—C(O)), 5.6 (s, 2H, ═C—H), 5.8 (t, 17H, —CF₂—H), 6.1 (m, 35H, —CF₂—H and ═C—H), and 6.3 (t, 17H, —CF₂—H).

The procedures of Example 2 are summarized below.

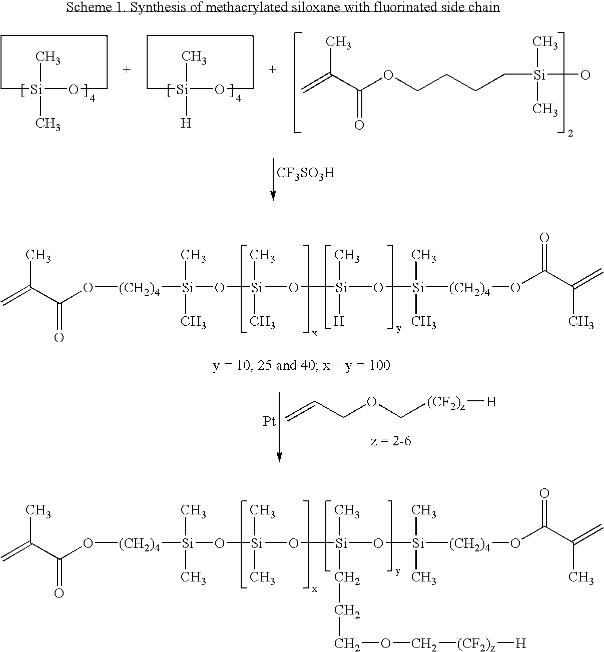

### Example 3

Fluorosilicone film casting/drug delivery device fabrication. To 30 parts of the methacrylate end-capped fluorinated side-chain polymer of Example 2 is added 70 parts by weight of methyl methacrylate and 0.5 weight percent of Darocur 1173™ UV initiator. Fluocinolone acetonide (FA) is added to the monomer mix at the desired concentration. If desired, acetone can be used as a solubilizing agent. The clear solution is sandwiched between two silanized glass plates using metal gaskets and cast into a film by exposure to UV radiation for two hours. The resultant films are released from the glass plates and exposed to a supercritical carbon dioxide extraction to remove the unreacted monomers. The films are exposed to borate buffered saline at 37° C.

### Example 4

Fluorosilicone film casting/drug delivery device fabrication. To 70 parts by weight of the methacrylate end capped fluorinated side chain polymer of Example 2 is added 30 parts by weight of dimethylacrylamide, 20 parts by weight of hexanol, 1.0 weight percent Irgacure 819™ photoinitiator, and 15 weight percent of FA. The clear solution is sandwiched between two silanized glass plates using metal gaskets and exposed to UV radiation for two hours. The resultant films are released from the glass plates and extracted using supercritical carbon dioxide to remove the unreacted monomers. The films are exposed to borate buffered saline at 37° C.

### Example 5

Fluorosilicone film casting/drug delivery device fabrication. To 70 parts by weight of the methacrylate end capped fluorinated side chain polymer of Example 2 is added 30 parts by weight of methyl methacrylate, 0.5 weight percent Vazo 64 thermal initiator and 10 parts by weight timolol maleate. The solution is sandwiched between two silanized glass plates using metal gaskets and polymerized thermally using a cure format of one hour at 60° C., one hour at 80° C. and one hour at 100° C. The resultant films are released and extracted using supercritical carbon dioxide to remove unreacted silicone and methacrylate monomer.

The examples and illustrated embodiments demonstrate some of the drug delivery device designs for which the present invention may be employed. However, it is to be understood that these examples are for illustrative purposes only and do not purport to be wholly definitive as to the conditions and scope. While the invention has been described in connection with various preferred embodiments, numerous variations will be apparent to a person of ordinary skill in the art given the present description, without departing from the spirit of the invention and the scope of the appended claims.

What is claimed:

1. A method for making an ocular drug delivery device, comprising:
providing a drug delivery device comprising a polymeric material and a pharmaceutically active agent, said polymeric material including contaminants, and said drug delivery device being sized and configured for implantation or injection in eye tissue; and
subjecting the device to a supercritical fluid to remove the contaminants.

2. The method of claim 1, wherein the contaminants include at least one member selected from the group consisting of unreacted monomers and oligomers.

3. The method of claim 1, wherein the polymeric material comprises a silicone-containing polymer.

4. The method of claim 1, wherein the supercritical fluid comprises carbon dioxide.

5. The method of claim 1, wherein the device comprises a drug core that includes the active agent, and a holder comprising the polymeric material, wherein the drug core is held in the holder.

6. The method of claim 5, wherein the holder includes at least one opening for passage of the pharmaceutically agent.

7. The method of claim 5, wherein the drug core comprises a mixture of the active agent and a permeable polymeric material that is permeable to said active agent.

8. The method of claim 7, wherein the permeable polymeric material comprises poly(vinyl alcohol) and the holder comprises a silicone-containing polymer.

9. The method of claim 5, wherein the holder comprises a cylinder that surrounds the drug core.

10. The method of claim 9, wherein the device includes a suture tab attached to said cylinder for suturing the device to eye tissue.

11. The method of claim 5, wherein the drug core is coated with a material permeable to said active agent.

12. The method of claim 1, wherein the device comprises a matrix of the polymeric material and the pharmaceutically active agent.

13. The method of claim 12, wherein the polymeric material comprises a silicone-containing polymer.

14. The method of claim 13, wherein the polymeric material comprises a fluorosilicone-containing polymer.

15. The method of claim 12, wherein the polymeric material comprises a silicone-containing hydrogel copolymer.

16. The method of claim 1, wherein the drug delivery device comprises a pharmaceutically active salt, and the contaminants are hydrophobic.

17. The method of claim 16, wherein the contaminants include at least one member selected from the group consisting of silicone-containing unreacted monomers and oligomers.

18. A method comprising:
providing an ocular drug delivery device, the drug delivery device comprising a polymeric material and a pharmaceutically active agent; and
removing contaminants from the device by subjecting the device to a supercritical fluid.
